(19) United States
(12) Patent Application Publication
(10) Pub. No.: US 2025/0329443 A1
(43) Pub. Date: Oct. 23, 2025

(54) MEDICAL INFORMATION COMMUNICATION APPARATUS AND MEDICAL INFORMATION COMMUNICATION METHOD

(71) Applicant: Canon Medical Systems Corporation, Otawara-shi (JP)

(72) Inventor: Hidenori TAKESHIMA, Tokyo (JP)

(73) Assignee: Canon Medical Systems Corporation, Otawara-shi (JP)

(21) Appl. No.: 19/082,646

(22) Filed: Mar. 18, 2025

(30) Foreign Application Priority Data
Apr. 18, 2024 (JP) 2024-067562
Jan. 31, 2025 (JP) 2025-015475

(51) Int. Cl. G16H 40/20; G16H 30/20; G16H 40/60

(52) U.S. Cl. CPC G16H 40/20; G16H 30/20; G16H 40/60

(57) ABSTRACT

According to one embodiment, a medical information communication apparatus includes processing circuitry. The processing circuitry is configured to select an imaging condition for acquiring medical data. The processing circuitry is configured to determine whether or not checking by an approver is required for the imaging condition. The processing circuitry is configured to send the imaging condition to a terminal configured to enable viewing by the approver if it is determined that the checking by the approver is required for the imaging condition.

### CROSS-REFERENCE TO RELATED APPLICATIONS

This application is based upon and claims the benefit of priority from Japanese Patent Application No. 2024-067562, filed Apr. 18, 2024, and Japanese Patent Application No. 2025-015475, filed Jan. 31, 2025, the entire contents of which are incorporated herein by reference.

### FIELD

Embodiments described herein relate generally to a medical information communication apparatus, a medical information communication method, and a medical information communication program.

### BACKGROUND

Acquisition of medical images with a medical image diagnostic apparatus requires a radiographer to select various imaging conditions, including a region of interest (a “region of interest” (ROI) or a “volume of interest” (VOI)), an imaging parameter, etc. Usually, doctors do not check the selected imaging conditions. If the selected imaging conditions are not appropriate, a doctor may fail to comprehend accurate information from the medical image or the medical data acquired under such imaging conditions, which could make a correct diagnosis result unobtainable.

As such, it is desirable to ask doctors to check imaging conditions as needed, but generally, an operating room where a radiographer operates the medical image diagnostic apparatus is often distant from the place doctors are located (a medical office, an examination room, or the like). Thus, there is a problem wherein it is difficult to have a doctor immediately check the selection of imaging conditions.

### BRIEF DESCRIPTION OF THE DRAWINGS

FIG. 1 is a block diagram showing a medical information communication system according to an embodiment.

FIG. 2 is a block diagram showing a requester-side communication apparatus.

FIG. 3 is a block diagram showing an approver-side communication apparatus.

FIG. 4 is a flowchart for explaining an exemplary operation of the requester-side communication apparatus.

FIG. 5 is a flowchart for explaining an exemplary operation of the approver-side communication apparatus.

FIG. 6 is a diagram showing a first example of query data.

FIG. 7 is a diagram showing a second example of the query data.

FIG. 8 is a diagram showing a third example of the query data.

FIG. 9 is a diagram showing an example of reply data.

FIG. 10 is a block diagram showing a medical information communication system according to Modification 1 of the embodiment.

FIG. 11 is a diagram showing an exemplary inquiry process in an instance where a medical image diagnostic apparatus according to Modification 2 is an X-ray computed tomography (CT) apparatus.

FIG. 12 is a diagram showing an exemplary inquiry process in an instance where the medical image diagnostic apparatus according to Modification 2 is an ultrasound diagnostic apparatus.

### DETAILED DESCRIPTION

In general, according to one embodiment, a medical information communication apparatus includes processing circuitry. The processing circuitry is configured to select an imaging condition for acquiring medical data. The processing circuitry is configured to determine whether or not checking by an approver is required for the imaging condition. The processing circuitry is configured to send the imaging condition to a terminal configured to enable viewing by the approver if it is determined that the checking by the approver is required for the imaging condition.

According to one embodiment, a medical information communication apparatus includes processing circuitry. The processing circuitry is configured to acquire an imaging condition selected for acquiring medical data, and a positioning image from a terminal operable by a requester. The processing circuitry is configured to send an indication of whether or not the imaging condition is corrected and, if the imaging condition is corrected, the corrected imaging condition to the terminal operable by the requester.

A medical information communication apparatus, a medical information communication method, and a medical information communication program according to one or more embodiments will be described with reference to the drawings. The description of the embodiments will assume that the components or portions having the same reference signs are adapted to operate in the same manner, and redundant explanations will be omitted as appropriate. A description of an embodiment will be given using the drawings.

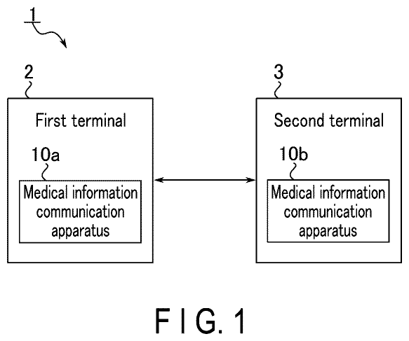

The block diagram in FIG. 1 will be referred to for describing a medical information communication system according to the embodiment. This medical information communication system, denoted by reference sign “1” here, includes a first terminal 2 and a second terminal 3. The first terminal 2 includes a medical information communication apparatus 10a. The second terminal 3 includes a medical information communication apparatus 10b.

For the following embodiments, the medical information communication apparatus 10a included in the first terminal 2 may also be called a “requester-side communication apparatus”, and the medical information communication apparatus 10b included in the second terminal 3 may also be called an “approver-side communication apparatus”.

The first terminal 2 is a terminal unit operable by a requester and is assumed to be, for example, a tomographic apparatus such as a magnetic resonance imaging (MRI) apparatus or an X-ray CT apparatus. The first terminal 2 requests the second terminal 3 to check one or more imaging conditions for a tomographic apparatus to acquire medical data. The imaging conditions here include the location and the size of a region of interest (ROI or VOI), and an imaging parameter. In one example where the tomographic apparatus is an MRI apparatus, the imaging conditions may include the location and the size of a region of interest for an MR image or an MR spectroscopy (MRS), a type of imaging sequence, TE, TR, a flip angle, items specific to MRS such as a position, an angle, a number used in outer volume suppression (OVS) or a number of excitations, and so on. If the tomographic apparatus is an X-ray CT apparatus, the imaging conditions may include a tube current, a tube voltage, a rotational rate of an X-ray tube, a slice thickness, a helical pitch, and so on. As the imaging conditions, any other items such as image resolution and contrast may also be adopted, as long as they serve as conditions (parameters) set for imaging.

The second terminal 3 is assumed to be a terminal which enables viewing by an approver and it may be any terminal such as a desktop PC, a laptop PC, a tablet terminal, or a smartphone that can present various types of information including images for viewing. The second terminal 3 displays one or more imaging conditions received from the first terminal 2 in such a form that can be checked by the approver and sends the result of checking by the approver to the first terminal 2.

The approver here refers to a senior rank person (expert) who checks and approves the selection of imaging conditions by a requester. Thus, in instances where the requester is a radiographer, the approver may be a doctor. Note that the requester-approver relationship may be applied to radiographers themselves or doctors themselves. In other words, the approver may be any person who can check the contents of imaging conditions. For example, supposing that there are a new radiographer and a supervising radiographer for this relationship, the requester is the new radiographer and the approver is the supervising radiographer.

Note that the requester may instead be a computer setting as represented by artificial intelligence (AI) or the like, so that, for example, imaging conditions may be select by the AI without human intervention. The approver in such a case is not limited and may be an radiographer, a doctor, or other human beings.

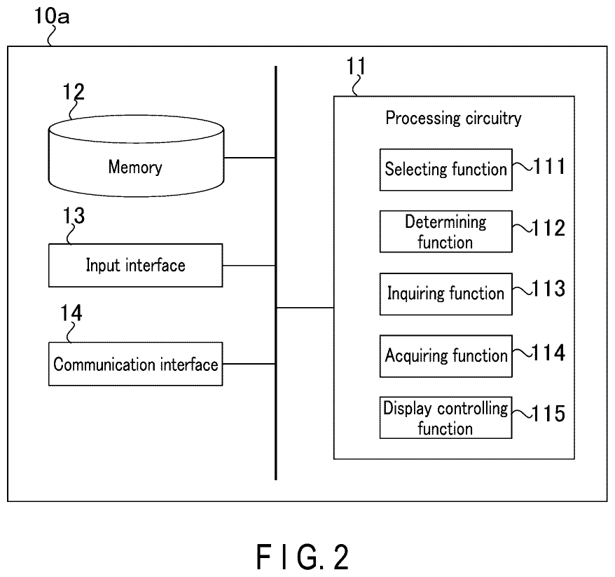

Next, the block diagram in FIG. 2 will be referred to for describing the requester-side communication apparatus (medical information communication apparatus 10a).

The requester-side communication apparatus may be, for example, a component included in a console (not shown in the figure) of the first terminal 2 in the instance where a tomographic apparatus is the first terminal 2, or independent from the first terminal 2 while being communicably coupled with the first terminal 2 and the second terminal 3.

The requester-side communication apparatus according to the embodiment includes processing circuitry 11, a memory 12, an input interface 13, and a communication interface 14.

The processing circuitry 11 includes a selecting function 111, a determining function 112, an inquiring function 113, an acquiring function 114, and a display controlling function 115. The processing circuitry 11 includes one or more processors (not shown in the figure) as hardware resources.

The selecting function 111 selects one or more imaging conditions for acquiring medical data.

The determining function 112 determines whether or not the checking by an approver is required for such imaging conditions.

The inquiring function 113 sends the imaging conditions to a terminal which enables viewing by the approver, if it is determined that the checking by the approver is required for the imaging conditions.

The acquiring function 114 acquires corrected imaging conditions which are the imaging conditions corrected by the approver.

The display controlling function 115 controls a display (not shown in the figure) of the first terminal 2 to display various information sets such as a positioning image, the imaging conditions, and the corrected imaging conditions.

The memory 12 stores various types of medical data and acquisition conditions, trained models, and so on described later. Examples of the memory 12 include a semiconductor memory device such as a random access memory (RAN) or a flash memory, a hard disk drive (HDD), a solid state drive (SSD), an optical disk, and so on. The memory 12 may also be a CD-ROM drive, a DVD drive, or a drive unit, etc., adapted to read and write various information sets from and to portable storage media such as a flash memory.

The input interface 13 includes circuitry for accepting inputs of various instructions or information sets from a user, etc. In one example, the input interface 13 includes circuitry pertaining to a pointing device such as a mouse, an input device such as a keyboard, or the like. Note that the circuitry included in the input interface 13 is not limited to circuitry pertaining to physical operational components such as a mouse and a keyboard. Examples of the input interface 13 also include processing circuitry for electric signals which receives an electric signal corresponding to an operational input from an external input device separate from the medical information communication apparatus and outputs this electric signal to various circuitry members in the medical information communication apparatus.

The communication interface 14 conducts wired and/or wireless data exchange between the first terminal 2 and the second terminal 3 via a network. For such a network, generally adopted communication means such as the Internet, Ethernet (registered trademark), and Bluetooth (registered trademark) may be utilized, and its explanations will be omitted.

The communication interface 14 is an interface which connects the first terminal 2 to a workstation, a picture archiving and communication system (PACS), a hospital information system (HIS), a radiology information system (RIS), etc., via a local area network (LAN), Ethernet, Bluetooth, or the like. The communication interface 14 communicates various information sets with the connected workstation, PACS, HIS, RIS, etc.

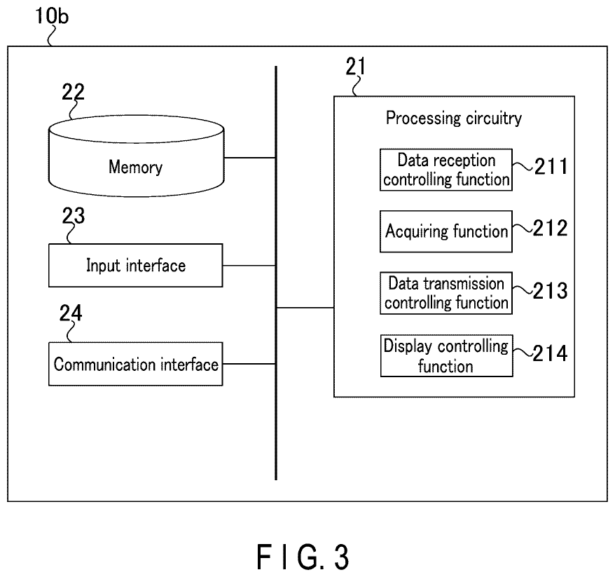

Next, the block diagram in FIG. 3 will be referred to for describing the approver-side communication apparatus (medical information communication apparatus 10b).

The approver-side communication apparatus according to the embodiment includes processing circuitry 21, a memory 22, an input interface 23, and a communication interface 24. The memory 22, the input interface 23, and the communication interface 24 may be of the same configuration as the memory 12, the input interface 13, and the communication interface 24 shown in FIG. 2, respectively, and their explanations will be omitted.

The processing circuitry 21 includes a data reception controlling function 211, an acquiring function 212, a data transmission controlling function 213, and a display controlling function 214. The processing circuitry 21 includes one or more processors (not shown in the figure) as hardware resources.

The data reception controlling function 211 performs control for receiving query data containing imaging conditions and a positioning image from the first terminal 2.

The acquiring function 212 acquires, based on the query data, the imaging conditions and the positioning image from the first terminal 2 operated by the requester.

The data transmission controlling function 213 performs control for sending, as reply data, an indication of whether or not a correction is made to the imaging conditions and, if a correction is made, the corrected imaging conditions to the first terminal 2.

The display controlling function 214 performs control for displaying the imaging conditions and the positioning image on a screen of the second terminal 3.

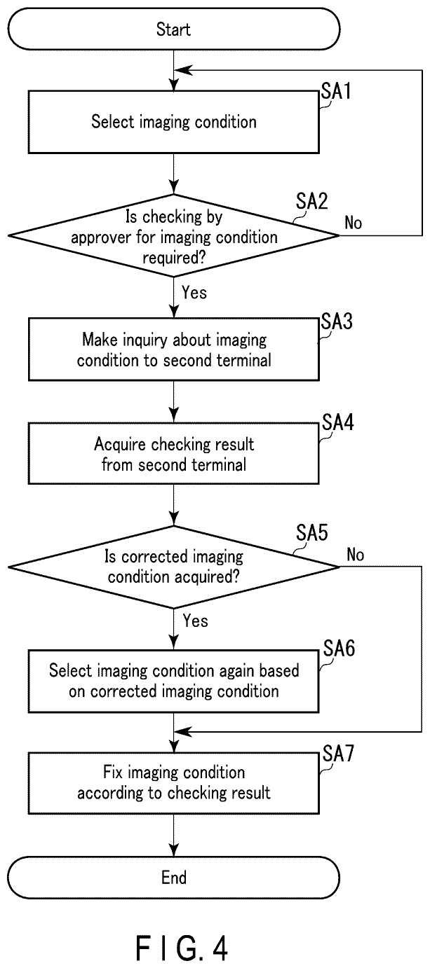

Next, an exemplary operation of the requester-side communication apparatus will be described with reference to the flowchart in FIG. 4.

In step SA1, the processing circuitry 11 with the selecting function 111 selects imaging conditions according to inputs by a user, i.e., a requester such as a radiographer.

In step SA2, the processing circuitry 11 with the determining function 112 determines whether or not the checking by an approver is required for the imaging conditions. As an example, a flag “Inquiry: Yes” may be prepared so that the determination that the checking by an approver is required for the imaging conditions is made if this flag is raised. On the other hand, if such a flag is absent, it may be determined that the checking by an approver is unnecessary. More specifically, it is possible to prepare a flag “Inquiry: Yes” for a stage of setting imaging protocols, and adopt a configuration in which if a requester raises the flag “Inquiry: Yes” in advance of inputting imaging conditions, the determination that the checking by an approver is required for the imaging conditions is made once the imaging conditions are select in step SA1. The processing circuitry 11 with the determining function 112 may determined that the checking for the imaging conditions by an approver is required if a particular imaging protocol is designated. For example, if there is a stipulation that the checking by an approver is mandatory for a region of interest in cases of MRS imaging protocols, the processing circuitry 11 with the determining function 112 may determine that the checking by an approver is required for the imaging conditions once MRS imaging conditions are select.

Note that the use of flags, such as flags representing two values “Inquiry: Yes” and “Inquiry: No”, is not a limitation. For example, representations of three values “Inquiry: Yes (with timeout)”, “Inquiry: Yes (no timeouts)”, and “Inquiry: No” may be used instead. In such an option, if the representation is “Inquiry: Yes (with timeout)”, a predetermined process, e.g., a process that assumes approval to the inquiry (or a process that assumes non-approval to the inquiry) may be advanced further upon elapse of a given period of time without a response. As such, in instances where additional information representing whether or not an inquiry should be made is set within, for example, imaging protocols, the processing circuitry 11 with the determining function 112 may determine, based on this additional information, whether or not the checking by an approver is required. If it is determined that the checking by an approver is required for the imaging conditions, the process proceeds to step SA3. If it is determined that the checking by an approver is not required for the imaging conditions, the process goes back to step SA1 and repeats the same processing contents until the next imaging conditions are select.

In step SA3, the processing circuitry 11 with the inquiring function 113 sends the imaging conditions with a flag to the second terminal 3 via a network so as to make an inquiry to an approver who is using the second terminal 3.

In step SA4, the processing circuitry 11 with the acquiring function 114 acquires a checking result for the imaging conditions from the second terminal 3.

In step SA5, the processing circuitry 11 with the determining function 112 determines whether or not corrected imaging conditions have been acquired as the checking result from the second terminal 3. If the corrected imaging conditions have been acquired, the process proceeds to step SA6. If the corrected imaging conditions have not been acquired, the process proceeds to step SA7.

In step SA6, the processing circuitry 11 with the selecting function 111 selects imaging conditions again based on the corrected imaging conditions.

In step SA7, the processing circuitry 11 with the selecting function 111 fixes the imaging conditions according to the checking result. If the checking result includes an indication that imaging is approved, the imaging conditions acquired in step SA1 are employed for carrying out imaging. If the imaging conditions have been select again in step SA6, such updated imaging conditions are fixed. If the checking result includes an indication that imaging is unnecessary or not to be performed, the imaging conditions acquired in step SA1 are not employed so that the imaging operation may be skipped.

Note that if a flag as discussed above is already raised before the selection of imaging conditions, the processing circuitry 11 with the inquiring function 113 may send a positioning image to the second terminal 3 before the selection of the imaging conditions. Also, with the above-discussed flag raised before the selection of imaging conditions, the processing circuitry 11 with the inquiring function 113 may send the imaging conditions to the second terminal 3 upon completion of the selection of the imaging conditions. In this manner, the positioning image, which is of a large data size, is sent to the second terminal 3 beforehand, and therefore, the second terminal 3 already has the positioning image by the time the imaging conditions are sent to the second terminal 3. Accordingly, a loss of time that might be incurred during a period from the selection of the imaging conditions up to the checking of the imaging conditions by an approver can be suppressed.

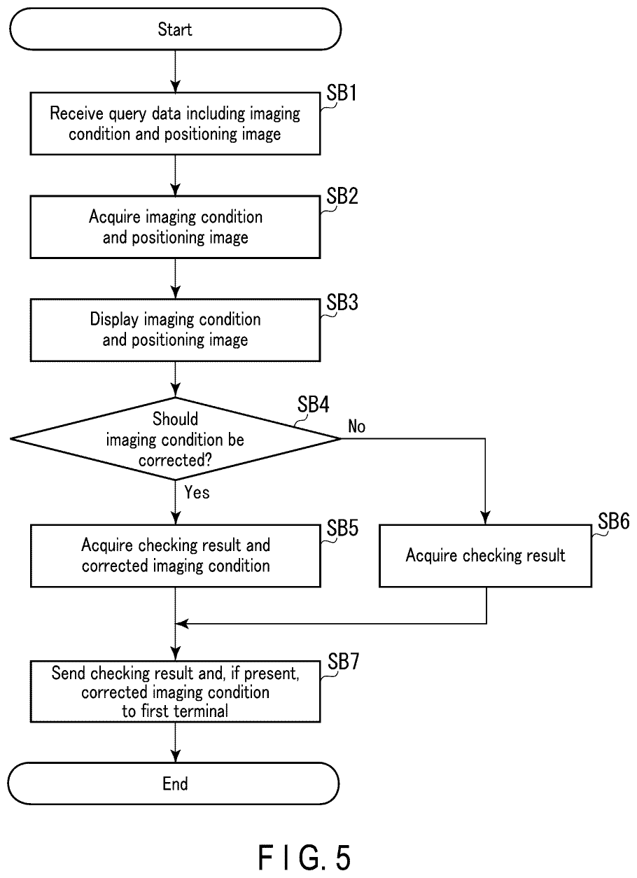

Next, an exemplary operation of the approver-side communication apparatus will be described with reference to the flowchart in FIG. 5.

In step SB1, the processing circuitry 21 with the data reception controlling function 211 receives query data, which will be described in detail later, relating to an inquiry about imaging conditions.

In step SB2, the processing circuitry 21 with the acquiring function 212 acquires imaging conditions and a positioning image to be checked by an approver by extracting them from the received query data. Here, for the positioning image, it is possible to adopt a setting in which the query data contains a link to enable downloading of the positioning image from, for example, a PACS server.

In step SB3, the processing circuitry 21 with the display controlling function 214 controls a display of the second terminal 3 to display the imaging conditions and the positioning image. As a method for displaying the imaging conditions and the positioning image, for example, a cross sectional image that a requestor deems necessary for the checking may be displayed by default. Also, if the imaging conditions involve a region of interest, three cross sections may be displayed for the positioning image in such a manner that the region of interest is superimposed for display. Display of the three cross sections for the positioning image may be automatically switched. As another form, one cross section may be displayed while being automatically scrolled. Further, the automatic scrolling may be started if an operational action from the user is unavailable for a preset period of time. The automatic scrolling may further be carried out sequentially in the x-axis direction, the y-axis direction, and the z-axis direction, one cross section by one cross section, and at a preset scrolling speed.

Note that, if, during the automatic scrolling, an operational input from the user is received, the automatic scrolling may be paused. Also, a resume button may be provided so that anytime the automatic scrolling is desired to be restarted, such a resume button may be pressed to carry out the automatic scrolling again.

In step SB4, the processing circuitry 21 determines, based on an input from an approver as the user, whether or not the imaging conditions should be corrected. For example, it is possible to adopt a configuration in which the determination that the imaging conditions should be corrected is made in response to the processing circuitry 21 with the acquiring function 212 acquiring the corrected value or the like of the imaging conditions from the approver. If the imaging conditions should be corrected, the process proceeds to step SB5. If no corrections are to be made to the imaging conditions, the process proceeds to step SB6.

In step SB5, the processing circuitry 21 with the acquiring function 212 acquires, based on an input from the approver as the user, a checking result which is the result of checking the imaging conditions, together with corrected imaging conditions.

In step SB6, the processing circuitry 21 with the acquiring function 212 acquires, based on an input from the approver as the user, a checking result which is the result of checking the imaging conditions.

In step SB7, the processing circuitry 21 with the data transmission controlling function 213 sends the checking result and, if generated, the corrected imaging conditions to the first terminal 2.

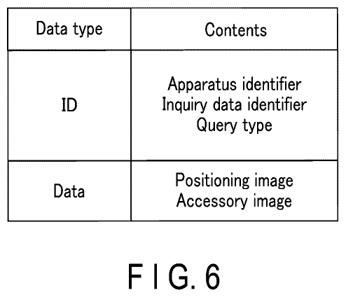

Next, with reference to FIGS. 6 to 8, a description will be given of data format examples of the query data relating to an inquiry about imaging conditions from the first terminal 2 to the second terminal 3.

FIG. 6 relates to query data for a positioning image and illustrates correspondence between data types and their respective data contents in the query data.

The data type “ID” includes, in its data contents, an apparatus identifier, an inquiry data identifier, and a query type. The apparatus identifier is, for example, an identifier that allows for unique identification of the apparatus for acquiring a positioning image. The inquiry data identifier is an identifier that allows for unique identification of associated inquiry data. The query type is an identifier that allows for unique identification of the query data.

The data type “Data” includes, in its data contents, a positioning image and accessory information. The positioning image is, in one example, a DICOM file series in which three-dimensional volume data may be included. This enables the approver to freely observe cross sections. The accessory information is, for example, parameter-related information for reproducing the imaging.

In instances where the approver-side communication apparatus receives query data for a positioning image as shown in FIG. 6 while it retains past query data for a positioning image that was sent from the same origin, the processing circuitry 21 with the data reception controlling function 211 may, based on the apparatus identifier, discard the past query data for a positioning image and instead save the query data for a positioning image that has been most recently received. As another option, the past query data may be kept without discarding and the received query data may be additionally saved.

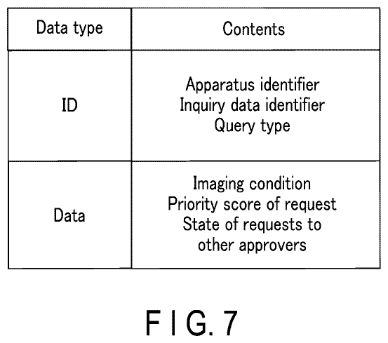

FIG. 7 relates to query data for imaging conditions and illustrates correspondence between data types and their respective data contents in the query data.

The data type “ID” includes, in its data contents, an apparatus identifier, an inquiry data identifier, and a query type, as in what is shown in FIG. 6. Here, in order to indicate that the positioning image and the imaging conditions correspond to each other, the same value is used for the inquiry data identifier as that for the inquiry data identifier in the query data for the positioning image.

The data type “Data” includes, in its data contents, imaging conditions, a priority score of a request to the approver, and a state of requests to other doctors. The imaging conditions include, if the item associated with the imaging conditions to be checked is, for example, a region of interest, a reference location (x-y-z coordinate information) of the region of interest, a reference direction (directional vector of each of the x-, y-, and z-axes), and a size of the region of interest. For the imaging conditions, types may be defined by XML Schema or Protobuf, etc., so that the imaging conditions are sent to the second terminal 3 as the data corresponding to the types. Also, in such definitions by XML Schema or Protobuf, etc., definitions for frequently used data may be provided as a basic set and vendor-specific information may be reflected as extensions of this basic set. For the basic set, as one example, XML Schema included in the MRD format prepared at the International Society for Magnetic Resonance in Medicine (ISMRM) may be employed. In this way, parameters that are substantially common to any vendors can be set.

Also, in the data type “Data”, a reason for sending the query data may be written. For example, information indicating why the imaging conditions are desired to be checked by an approver may be included. In some instances, the data type “Data” may include an entry which permits a free description such as a question based on a hypothesis using different values, etc.

Note that if the approver-side communication apparatus receives query data for a region of interest shown in FIG. 7, the processing circuitry 21 with the data reception controlling function 211 determines whether or not the approver-side communication apparatus stores query data for a positioning image that includes an inquiry data identifier identical to that of the received query data for the region of interest. If the approver-side communication apparatus does not store query data for a positioning image that includes the identical inquiry data identifier, a request may be made to the requester-side communication apparatus so that such query data for a positioning image that includes the identical inquiry data identifier will be provided.

Also, upon both of the query data for a positioning image and the query data for the region of interest properly becoming ready, the processing circuitry 21 with the display controlling function 214 in the approver-side communication apparatus controls the display of the second terminal 3 to provide a display presentation which enables the approver to confirm the state. In other words, a state where only one of the query data for a positioning image and the query data for a region of interest is in receipt is deemed to represent a shortage of information for the approver to check the imaging conditions. As such, providing a display presentation upon both the positioning image data and the region of interest data becoming ready can contribute to the efficient checking of the imaging conditions by the approver.

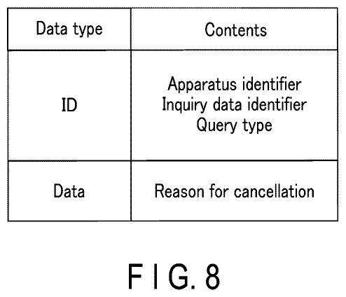

FIG. 8 relates to canceling query data for canceling a query that has been sent to an approver for inquiry, and illustrates correspondence between data types and their respective data contents.

The data type “ID” includes, in its data contents, an apparatus identifier, an inquiry data identifier, and a query type, as in what is shown in FIG. 6.

The data type “Data” includes, in its data contents, a reason for cancellation. For example, in instances where positioning image data or a region of interest for inquiry involves an error, or a targeted entity for inquiry is mistaken, such reasons are indicated.

Note that each piece of query data shown in FIGS. 6 to 8 is only an example, and the query data may be generated in any format or constitution where, for example, information sets associated with a positioning image and imaging conditions may be formulated into one piece of query data.

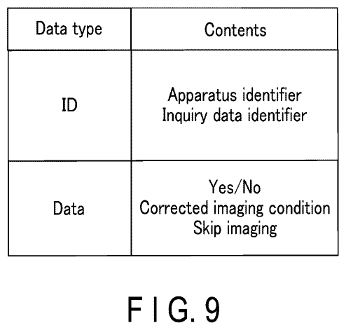

Next, FIG. 9 will be referred to for describing an exemplary data format of reply data sent from the approver-side communication apparatus to the requester-side communication apparatus.

FIG. 9 is a diagram illustrating correspondence between data types and their respective data contents in the reply data.

The data type “ID” includes, in its data contents, an apparatus identifier and an inquiry data identifier, of which values may be copied from the corresponding query data.

The data type “Data” includes, in its data contents, any of the information items “Yes”, which indicates approval for the imaging under imaging conditions included in the query data, “No”, which indicates non-approval for the imaging under the imaging conditions, “Corrected imaging condition”, which includes a value or values of the imaging conditions corrected by the approver, and “Skip”, which indicates that the imaging action itself is to be halted (skipped).

In the case where checking by more than one approver is required, the order of sending the set of query data for a positioning image and query data for imaging conditions may be determined according to priority scores. For example, the query data set is sent to the second terminal 3 that is in use by an approver with the highest priority, based on the priority score stored in the data as shown in FIG. 7 for the request to the approver. Once the reply data to this query data set is acquired, the query data set may be sent to another second terminal 3 that is in use by an approver with the next highest priority.

If the requester-side communication apparatus is unable to receive a checking result from the first approver for a predetermined period of time, the requester-side communication apparatus sends canceling query data to the first approver and sends query data for the positioning image and the imaging conditions to the second approver different from the first approver.

In the requester-side communication apparatus, the processing circuitry 11 with the acquiring function 114 acquires the reply data. If it is indicated that the approver has determined the imaging conditions to be non-problematic, imaging is conducted using the imaging conditions as they are. On the other hand, if the imaging conditions have been corrected by the approver, the processing circuitry 11 with the selecting function 111 selects imaging conditions again based on the corrected imaging conditions and imaging is conducted. If the reply data indicates a skip instruction, imaging may be skipped.

There may be instances where a single exchange of query data and reply data between the requester and the approver does not provide a satisfactory result and more than one exchange would be required. In such instances, a serial number or the like may be added to the ID in the query data. The reply data from the approver may be provided with a draft flag indicating a draft stage so that the imaging conditions may be determined to be in a not-yet-fixed state while the draft flag is up. The requester-side communication apparatus which has received this reply data may again send the query data with the serial number incremented by one to the approver-side communication apparatus.

In this manner, while the imaging conditions are not fixed, the presence of the flag allows the requester-side communication apparatus to easily comprehend the not-yet-fixed state of the imaging conditions even in the course of ongoing exchange of the query data and the reply data. Then, the approver-side communication apparatus, once it has fixed (committed to) the imaging conditions, may delete the draft flag and send the reply data to the requester-side communication apparatus. Note that the form of a draft flag is not a limitation and any form of additional information, such as a description by a natural sentence, may be employed as long as it can indicate a not-yet-fixed state of imaging conditions.

Also, a link or the like may be incorporated into the query data and the reply data so that the requester and the approver can interact with each other. For example, the data portions in the query data and the reply data may include a link for running an application that enables chatting, IP telephone, video meeting, etc. This allows the requester or the approver to make instant communications with the other party via the link.

### Modification 1

The foregoing embodiment has assumed a configuration in which the requester-side communication apparatus is included in the first terminal 2 and the approver-side communication apparatus is included in the second terminal 3 for conducting mutual communications. However, for example, it is also possible to adopt a configuration in which a medical information communication apparatus is included in an external server such as a PACS server, and the first terminal 2 and the second terminal 3 access the external server so as to carry out inquiry and checking processes for imaging conditions.

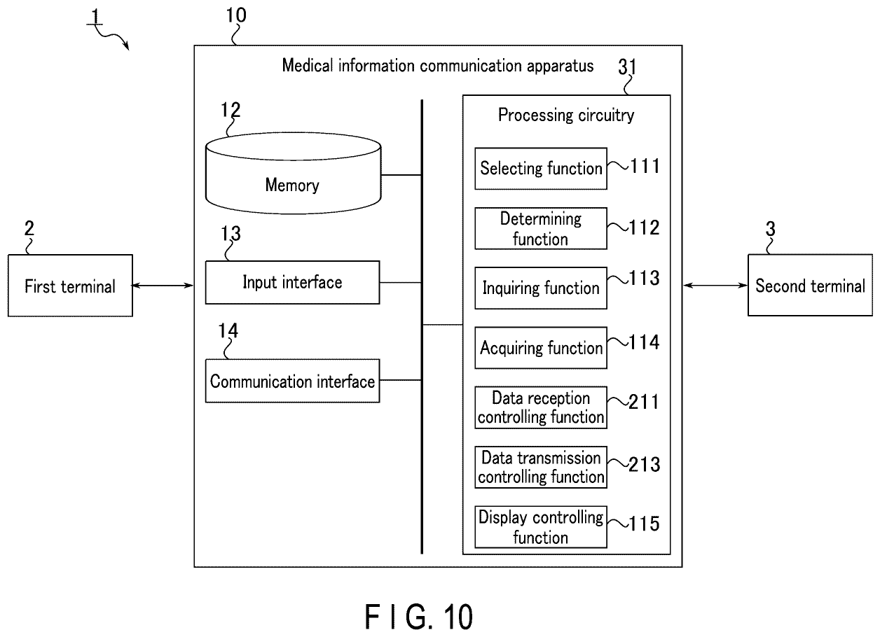

The block diagram in FIG. 10 will be referred to for describing a medical information communication apparatus 10 according to Modification 1 of the embodiment.

A medical information communication system 1 shown in FIG. 10 includes a first terminal 2, the medical information communication apparatus 10, and a second terminal 3. The medical information communication apparatus 10 is independent from the first terminal 2 and the second terminal 3. The medical information communication apparatus 10 includes processing circuitry 31, a memory 12, an input interface 13, and a communication interface 14. The processing circuitry 31 includes a determining function 112, an inquiring function 113, an acquiring function 114, a data reception controlling function 211, a data transmission controlling function 213, and a display controlling function 115.

The functions of the processing circuitry 31 are equivalent to the respective functions shown in FIGS. 2 and 3. The medical information communication apparatus 10 according to Modification 1 acquires selected imaging conditions from the first terminal 2. The medical information communication apparatus 10, if checking of the imaging conditions is determined to be necessary, performs control so that the second terminal 3 displays the imaging conditions and a positioning image. The medical information communication apparatus 10 acquires, from an approver who views the imaging conditions and the positioning image through the second terminal 3, inputs of a checking result for the imaging conditions and, as needed, corrected imaging conditions to select for the first terminal 2.

Note that in instances where the requester is a computer setting such as an AI entity and the approver is a human such as a radiographer, the first terminal 2 and the second terminal 3 may be the same single device. Here, communications for transmission and reception may take the form of an inter-process communication (IPC).

### Modification 2

As Modification 2 of the embodiment, another example of the inquiry process will be described assuming that the first terminal 2 is any of the various medical image diagnostic apparatuses including an X-ray CT apparatus, an MRI apparatus, an ultrasound diagnostic apparatus, and so on.

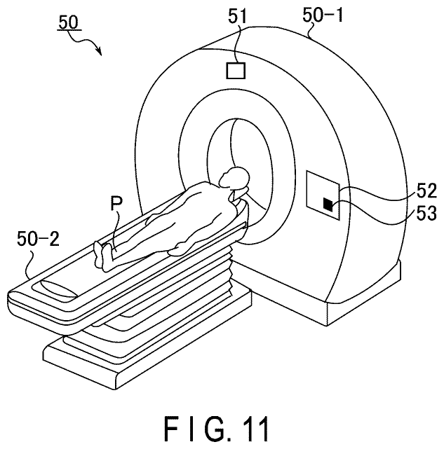

FIG. 11 shows an exemplary inquiry process in an instance where the medical image diagnostic apparatus according to Modification 2 is an X-ray CT apparatus. This X-ray CT apparatus 50 includes a gantry unit 50-1 and a couch 50-2. The gantry unit 50-1 is furnished with a visible light camera 51 and an operation panel 52. The visible light camera 51 is arranged so as to be capable of imaging a subject P placed on the couch 50-2. Note that a connection between the visible light camera 51 and the gantry unit 50-1 is not mandatory but the visible light camera 51 may be arranged on the sealing in the examination room or at any location that allows for the imaging of the subject P. The operation panel 52 of the gantry unit 50-1 is provided with a query button 53. The query button 53 may be either a virtual button formed on a touch display or a physical button.

For example, what is inquired about by a requester is not limited to imaging conditions as in the foregoing embodiment, etc., but the requester may inquire about the setting state of the subject P. More specifically, the requester may, after the setting for imaging the subject P with the X-ray CT apparatus is done, press the query button 53. In response to the query button 53 being pressed, the processing circuitry 11 with the inquiring function 113 sends an image of the subject P acquired by the visible light camera 51 to the second terminal 3. The second terminal 3 displays the image acquired by the visible light camera 51. This enables an approver to determine whether or not the subject P is appropriately set.

Note that, while the description here assumes an example of the X-ray CT apparatus 50, the visible light camera 51 and the query button 53 may likewise be arranged for an MRI apparatus as in the case of the X-ray CT apparatus 50. For a use case, as one example, if the knee of a subject P is to be imaged with an MRI apparatus, pressing of the query button 53 may trigger the transmission of an image taken by the visible light camera 51 to the second terminal 3 so that an approver can check whether or not the lateral position of the receiving coil set above the subject P is appropriate.

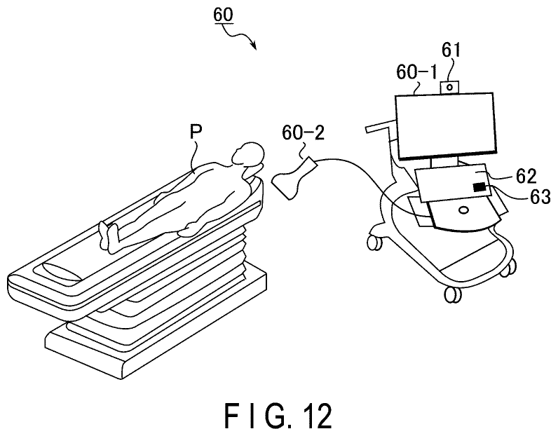

FIG. 12 shows an exemplary inquiry process in an instance where the medical image diagnostic apparatus according to Modification 2 is an ultrasound diagnostic apparatus.

This ultrasound diagnostic apparatus 60 includes an ultrasound diagnostic apparatus main unit 60-1 and an ultrasound probe 60-2. The ultrasound diagnostic apparatus main unit 60-1 is furnished with a visible light camera 61 and an operation panel 62. The operation panel 62 is provided with a query button 63. The visible light camera 61 and the query button 63 serve the purpose as long as they are provided in a similar arrangement state and have similar functions as those discussed with reference to FIG. 11.

As one example, the description will assume an instance where a requester is presently conducting examination of a subject P using the ultrasound diagnostic apparatus 60 and ultrasound images are acquired in real time. In response to the requester pressing the query button 63, the processing circuitry 11 with the inquiring function 113 sends an ultrasound image at the time point concurrent with the pressing of the query button 63 to the second terminal 3.

Through the second terminal 3, an approver can determine whether or not the ultrasound image is a desirable image. The requester may continue the examination with the ultrasound diagnostic apparatus 60 even after the pressing of the query button 63. Here, if a checking result indicating that the ultrasound image sent as a request is not a desirable image is returned from the second terminal 3, the requester may again acquire an ultrasound image of a subject site or a subject.

Moreover, the inquiry process according to the embodiment may also be utilized for checking the skills of and coaching a radiographer, i.e., the requestor, with the ultrasound probe 60-2. For example, the visible light camera 51 is capable of taking moving images, and it acquires a moving image that focuses on the movement of the ultrasound probe 60-2 by the radiographer during examination. In response to the requester pressing the query button 63, the processing circuitry 11 with the inquiring function 113 sends a moving image covering a range from the start of the imaging action to the pressing of the query button 63 to the second terminal 3. An approver checks the moving image so that the approver can determine whether or not the skills being exhibited by the radiographer with the ultrasound probe 60-2 according to the examination are satisfactory for acquiring a desirable ultrasound image.

According to the one or more embodiments, etc. described above, a medical information communication apparatus, in instances where imaging conditions need to be checked by an approver, sends the imaging conditions and a positioning image to a device which enables viewing by the approver. The approver can check the imaging conditions in their free time by reviewing the imaging conditions through, for example, a laptop PC, a tablet terminal, or a similar device.

For example, in instances where a radiographer wishes a doctor to check imaging conditions, the imaging conditions may be directly sent to a tablet terminal or the like so as to entrust the doctor with the checking, even if the doctor is far away. Therefore, the checking of the imaging conditions by the approver can be smoothly carried out while the burden on the approver is reduced.

Note that the term “processor” or the like used herein refers to, for example, a central processing unit (CPU) or a graphics processing unit (GPU), or various types of circuitry which may be an application-specific integrated circuit (ASIC), a programmable logic device (such as a simple programmable logic device (SPLD), a complex programmable logic device (CPLD), or a field programmable gate array (FPGA)), and so on. If, for example, the processor is a CPU, the processor reads and executes a program or programs stored in storage circuitry to realize the functions. If, for example, the processor is an ASIC, the functions are directly incorporated into circuitry of the processor in the form of a logic circuit, instead of the corresponding programs being stored in storage circuitry. Each processor in the embodiments, etc. is not limited to a single circuit-type processor, and multiple independent circuits may be combined and integrated as one processor to realize the functions. Furthermore, multiple components or features as given in the figures may be integrated into one processor to realize the respective functions.

Moreover, the functions according to the embodiments, etc. may also be realized by installing a program or programs for executing the described processes in a computer such as a work station, and loading the program or programs in the memory. Here, the program or programs for causing the computer to execute the processes may be stored in a storage medium such as a magnetic disk (a hard disk, etc.), an optical disk (a CD-ROM or a DVD, etc.), or a semiconductor memory, and distributed.

While certain embodiments have been described, these embodiments have been presented by way of example only, and are not intended to limit the scope of the inventions. Indeed, the novel embodiments described herein may be embodied in a variety of other forms. Furthermore, various omissions, substitutions, and changes in the form of the embodiments may be made without departing from the spirit of the inventions. The accompanying claims and their equivalents are intended to cover such forms or modifications as would fall within the scope and spirit of the inventions.

What is claimed is:

1. A medical information communication apparatus comprising processing circuitry configured to:
select an imaging condition for acquiring medical data;
determine whether or not checking by an approver is required for the imaging condition; and
send the imaging condition to a terminal configured to enable viewing by the approver if it is determined that the checking by the approver is required for the imaging condition.

2. The medical information communication apparatus according to claim 1, wherein the processing circuitry is configured to, if additional information indicating that the checking by the approver is required is set, send the imaging condition to the terminal once selection of the imaging condition is completed.

3. The medical information communication apparatus according to claim 2, wherein the processing circuitry is configured to send a positioning image to the terminal before the selection of the imaging condition, the positioning image being acquired in advance of acquiring the medical data.

4. The medical information communication apparatus according to claim 1, wherein the processing circuitry is configured to, if checking by more than one approver is required for the imaging condition, send the imaging condition to the more than one approver in an order according to priority scores.

5. The medical information communication apparatus according to claim 1, wherein the processing circuitry is further configured to acquire a corrected imaging condition which is the imaging condition corrected by the approver, wherein the processing circuitry is configured to select a new imaging condition again based on the corrected imaging condition.

6. The medical information communication apparatus according to claim 1, wherein the imaging condition includes a region of interest.

7. The medical information communication apparatus according to claim 1, wherein the processing circuitry is configured to wait for user input indicating that whether or not checking by the approver is required.

8. A medical information communication apparatus comprising processing circuitry configured to:
acquire an imaging condition selected for acquiring medical data, and a positioning image from a terminal operable by a requester; and
send an indication of whether or not the imaging condition is corrected and, if the imaging condition is corrected, the corrected imaging condition to the terminal operable by the requester.

9. The medical information communication apparatus according to claim 8, wherein the processing circuitry is further configured to set additional information to the corrected imaging condition, the additional information indicating that checking of an imaging condition that is selected again based on the corrected imaging condition is required.

10. The medical information communication apparatus according to claim 8, wherein the imaging condition includes a region of interest.

11. A medical information communication method comprising:
selecting an imaging condition for acquiring medical data;
determining whether or not checking by an approver is required for the imaging condition; and
sending the imaging condition to a terminal configured to enable viewing by the approver if it is determined that the checking by the approver is required for the imaging condition.
